(19) United States
(12) Patent Application Publication
(10) Pub. No.: US 2005/0266542 A1
(43) Pub. Date: Dec. 1, 2005

(54) Methods for refining concentrated enzyme solutions

(76) Inventors: Dieter Baur, Vilassar de Mar (ES); Werner Pichler, Kundl (AT); Wilfried Raehse, Duesseldorf (DE); Jens Van Holt, Kundl (AT)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/194,333

(22) Filed: Aug. 1, 2005

Related U.S. Application Data
(63) Continuation of application No. PCT/EP04/00551, filed on Jan. 23, 2004.

(30) Foreign Application Priority Data
Jan. 31, 2003 (DE) 103 04 066.8

(51) Int. Cl. C12N 9/00

(52) U.S. Cl. 435/183

(57) ABSTRACT

Concentrated enzyme solutions are refined by a process comprising the steps of: (A) providing a concentrated enzyme solution comprised of insoluble solids; (B) separating the insoluble solids to produce a solids-free supernatant solution and (C) contacting the supernatant solution with a strongly basic anion exchanger carried out with a bed volume of from 1 to 10 to produce a decolorized protein solution. No precipitation takes place in the process. Instead, the target enzymes remain in solution throughout the entire process, for which purpose certain concentration ranges are particularly advantageous in regard to the yield to be obtained.

### CROSS REFERENCE TO RELATED APPLICATIONS

This application is a continuation under 35 U.S.C. § 365(c) and 35 U.S.C. § 120 of international application PCT/EP2004/000551, filed Jan. 23, 2004. This application also claims priority under 35 U.S.C. § 119 of DE 103 04 066.8, filed Jan. 31, 2003, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

This invention relates to processes for refining concentrated industrial enzyme solutions, to the products obtained by this process and to compositions based on such solutions, more particularly detergents and cleaning compositions.

Nowadays, enzymes, particularly those for industrial applications, are mostly produced by fermentation of microorganisms and are then purified from the media used. The concentrated enzyme solutions usually obtained by means of several sequential process steps are often also referred to as “liquid enzyme.” Liquid enzyme may be regarded as a purified raw material which is either used in liquid form or—occasionally—is converted into a dry form and then put to appropriate uses.

Important industrial applications for enzymes, particularly in liquid form, are detergents and cleaning compositions which are increasingly being marketed in liquid or gel form. Other applications include, for example, the cosmetics field, where the enzymes are used as active agents, as they are in detergents and cleaning compositions, the manufacture and processing of textiles and food production where, primarily, the raw materials are converted into the end product by the use of the enzymes.

Purification or enrichment processes for obtaining concentrated enzyme solutions are described in detail in the prior art. Important objectives in this regard are the removal of the biomass, i.e., the constituents of the host organisms, more particularly macromolecular constituents, the removal of low molecular weight accompanying substances and impurities, more particularly media constituents and metabolites, and the removal of other proteins, more particularly enzymes. At the same time, the target product is intended to be obtained in large quantities, in high purity and with a high activity level. On the other hand, the culture supernatants generally contain factors, often peptides or proteins, whose identity is often still unknown, but which provide for stabilization of the target enzyme. Accordingly, it is of particular advantage to obtain an enzyme solution which is not completely pure, i.e., contains a certain percentage of such stabilizing factors.

(2) Description of Related Art, Including Information Disclosed Under 37 C.F.R. §§ 1.97 and 1.98

Techniques based on filtration, sedimentation or precipitation are generally used for purification.

For example, processes generally applied in succession have been developed for the removal of biomass and are now established in the prior art. Such processes include, for example, separation, microfiltration and ultrafiltration. Only thereafter is it actually possible to speak of an enzyme concentrate in the context of the present invention.

For example, International Patent Application WO 01/37628 A2 describes a process for recovering biotechnologically produced useful materials from culture and/or fermenter solutions, which comprises separating the water-insoluble solids from the aqueous solution containing the useful materials, subsequently filtering the solution obtained and concentrating the solution containing the useful materials by ultrafiltration. This known process is characterized in that the solids removed are subjected to a washing step using the filtrate from the concentration step as the washing liquid.

However, there has as yet been no solution to the problem that enzyme concentrates removed from the biomass contain, in particular, the following impurities:
1. Solids, more particularly precipitates of irreversibly denatured proteins, including the target protein;
2. Colored, mostly brown compounds which are formed during the pre-fermentation sterilization of the media constituents, more particularly the nitrogen sources (Maillard compounds); and
3. Factors which increase the stability of the target protein.

Conventional refining processes are not sufficiently suitable for removing denatured proteins and colored compounds from an enzyme concentrate separated from the biomass or for removing a large part of the stabilizing factors together with the denatured proteins and colored compounds. In either case, the result is unsatisfactory product quality: either the enzyme concentrate is dark in color, striated and/or contains suspended matter up to and including precipitated substances, or it is light in color and forms a clear solution, but has unsatisfactory enzyme stability, which, in general, can only be improved to a certain extent by the addition of (expensive) stabilizing compounds. These disadvantages affect above all the products in which the concentrate in question is incorporated.

For example, liquid detergents and cleaning compositions should contain a high percentage of active, i.e., stable, enzyme throughout the storage period. However, water content and stability are inversely proportional to one another. At the same time, the compositions in question should have a color attractive to the consumer and a clear appearance.

Processes for decoloring concentrated enzyme solutions are described in the prior art. They include precipitation processes, for example using organic solvents or polymers, but more especially the salting out of the target protein with sodium sulfate (described in H. Ruttloff (1994): “Industrielle Enzyme”, Behr's Verlag, Hamburg, Chapter 6.3.3.6, pages 376 to 379). For example, United States patent U.S. Pat. No. 5,405,767 discloses certain compounds which are said to be added to the protein solution to be precipitated in order to obtain advantageous precipitates. When the protein is precipitated, the accompanying substances remain in the supernatant. However, part of the protein is irreversibly denatured and, overall, stability is impaired by the precipitation and re-suspension, not the least because stabilizing compounds are removed. A figure of ca. 50% is mentioned as the yield to be obtained in the TABLE on page 378 of the cited textbook.

Another alternative is adsorptive purification of the enzymes, for example using an ion exchange resin (H. Ruttloff (1994): “Industrielle Enzyme”, Behr's Verlag, Hamburg, Chapters 6.3.3.7 and 6.3.3.8, pages 379 to 396). In this process, the target proteins bind to a chromatography material and are then eluted with another medium. However, the yields obtained are again generally poor as a result of denaturing and folding effects. Thus, yields of at most 60% are mentioned for various chromatography processes (except affinity chromatography) in the TABLE on page 378. Although the specific chromatography materials (more particularly affinity chromatography materials) are generally more effective, they are very sensitive and expensive to make. Accordingly, the affinity chromatography materials are mainly used in analysis and in the medical field, but hardly at all in enzyme production on an industrial scale.

For example, patent application WO 89/05863 A1 describes the recovery of extracellular enzymes from the fermentation broth of Bacillus strains. This document describes experiments for removing cell wall polymers by ion exchange chromatography, more particularly in the preparation of amylase. The enzyme- and polymer-containing solution is first applied to the column, then rinsed with buffer and subsequently eluted with an elution medium. In other words, the alpha-amylase to be purified initially binds to the chromatography material in the same way as the accompanying substances and is only washed down thereafter as the ionic strength is increased.

The opposite approach, i.e., selectively removing the impurities from the liquid solution by means of a carrier material, has so far only been selected for food-grade raw materials. Thus, United States patent U.S. Pat. No. 5,972,121 discloses the removal of dyes from sugar solutions by means of weakly acidic or weakly basic adsorption chromatography. The decoloring of sugar, inter alia, by means of successive, different ion exchange chromatography steps is described in the manual “DIAION®. Manual of ion exchange resins and synthetic adsorbent, Vol. II,” Mitsubishi Kasei Corp. (Tokyo, Japan), 2nd printing, 1.5.1993, pages 93 to 100. This opposite approach basically involves several, each highly selective purification steps in which the particular removable impurity remains on the corresponding material.

United States patent U.S. Pat. No. 5,565,348, which is concerned with the recovery of a certain alkaline protease of a Bacillus, contains an EXAMPLE which describes the purification of that enzyme by a purification process consisting of several steps. These include, in particular, the steps of precipitation of the protein by salting out, taking up in another medium and dialysis before ion exchange chromatography is carried out. This is followed by affinity chromatography, i.e., a chromatography step in which the protease binds specifically to the column material. With regard to the ion exchange chromatography step, the document in question states that the special protease described does not bind to the special chromatography material used and is thus obtained with the break-through. However, this does not appear to be a generally applicable teaching for purification of the enzyme because the particular column material is not specified, the concentration of the enzyme is not mentioned and the actual removal of impurities took place in the preceding precipitation step or is guaranteed by the subsequent affinity chromatography. Accordingly, purifying an enzyme, more particularly a protease, by means of a column material to which it does not itself bind at all, has not hitherto been considered in the prior art, above all not without a precipitation step.

Accordingly, the problem addressed by the present invention was to free enzyme concentrates from solids, more particularly irreversibly denatured proteins, and to decolor the concentrates so selectively that they remain highly stable in storage.

### BRIEF SUMMARY OF THE INVENTION

This problem has been solved by the processes according to the invention, which comprise the following steps:
(A) Providing a concentrated enzyme solution comprised of insoluble solids;
(B) Separating the insoluble solids to produce a solids-free supernatant solution; and
(C) Contacting the supernatant solution with a strongly basic anion exchanger carried out with a bed volume of from 1 to 10 to produce a decolorized protein solution.

In the process according to the invention, no precipitation takes place in the refining of concentrated enzyme solutions. Instead, the target enzyme proteins remain in solution throughout the entire process, for which purpose, certain concentration ranges are particularly advantageous in regard to the yield to be obtained, as explained below and in the EXAMPLES.

### BRIEF DESCRIPTION OF THE SEVERAL VIEWS OF THE DRAWING(S)

FIG. 1 is a block flow chart of the refinement of concentrated enzyme solutions according to the invention.

FIG. 2 is a graph of the dependence of the solids content on the concentration of active enzyme as determined in EXAMPLES 1 and 2.

FIG. 3 is a graph of the storage stability of the protease in a liquid detergent matrix as determined in EXAMPLE 3 expressed as a graph of the residual activity as a function of storage time in weeks.

### DETAILED DESCRIPTION OF THE INVENTION

Process step (A) is preceded by processes known per se—described in the prior art—for the preparation of enriched, aqueous enzyme solutions substantially free from biomass. In general, this involves several component steps, such as cell break up, pelleting of the cell debris, decantation and optionally further centrifuging steps. Separation, microfiltration, ultrafiltration or sterile filtrations (see below) and concentration, i.e., removal of the solvent to a medium concentration range of the enzyme, may also be used. An enzyme concentration value of the order of half the range described as the optimal working concentration for the rest of the process (see below) may be regarded as optimal. Also advantageous is a target suspended particle or solids content of less than 1% by volume which can be verified, for example, by centrifuging for 10 mins. at 7,000 G in a tabletop centrifuge. In addition, the particular solution should be adjusted to a pH which is tolerable for the enzyme and at which it has a positive charge.

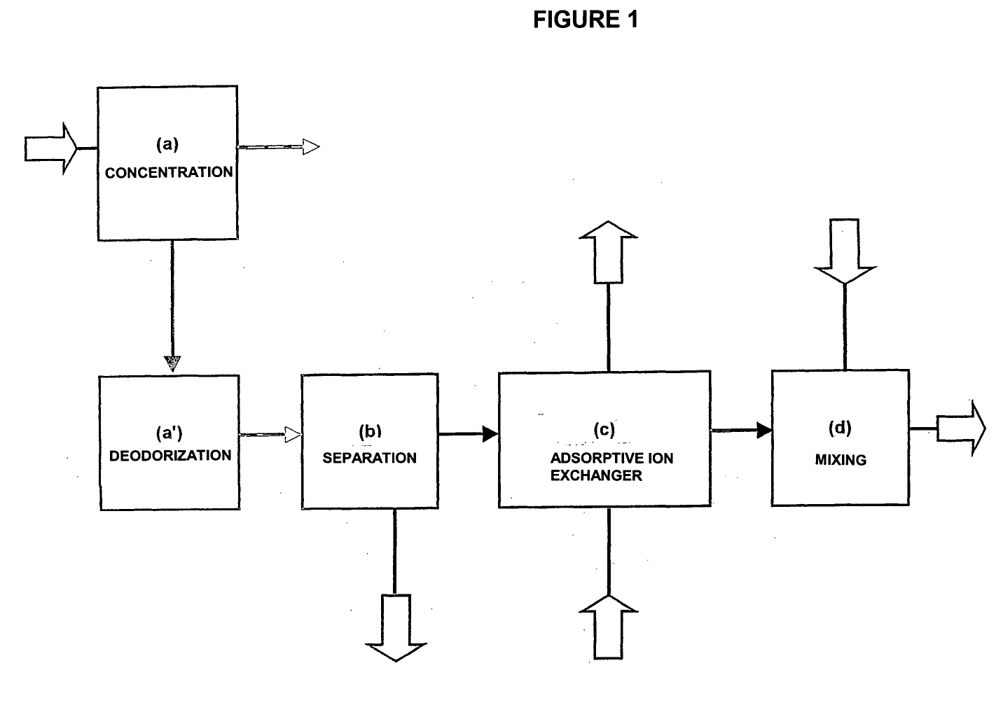

Step (A), which is designated as “concentration” in FIG. 1, is also based on processes known per se to the expert. A rotary evaporator or a thin-layer evaporator, for example, may be used for concentration. In a particularly advantageous embodiment, the pH remains substantially constant at the value adjusted beforehand, while the solids content of the enzyme concentrate remains as low as possible (see below). Otherwise, the losses in the following step (B) would undergo a considerable, unwanted increase.

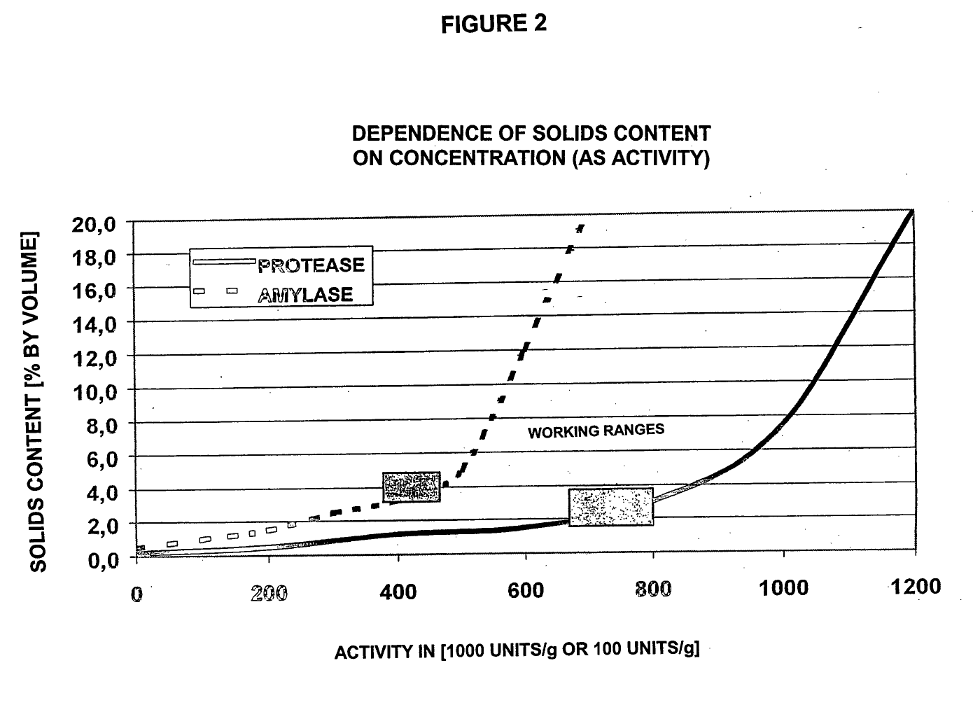

Step (A) should be controlled in known manner (in particular by stopping the concentration process at the right time) in such a way that the enzyme concentrate obtained continues to show no (quantitative) protein precipitation. The optimal working range has to be individually determined for each enzyme not only in regard to temperature, pH and ionic strength, but in particular in regard to an optimal enzyme concentration range. This was done in EXAMPLES 1 and 2 of the present application for the alkaline protease from Bacillus lentus and for the α-amylase from Bacillus sp. A 7-7 (DSM 12368). Accordingly, an optimal concentration range of 700,000 to 800,000 HPU/g was determined for the alkaline protease and one of 35,000 to 45,000 TAU/g for the α-amylase. Above these values, the percentage of solids precipitated soon increases overproportionally in dependence upon the activity which is accompanied by a dramatically increasing loss of useful product. These effects are illustrated in FIG. 2 for the enzymes mentioned by way of example. Such a dependence of the solids content on the concentration of protein and particularly on the concentration of enzyme activity is to be expected for virtually all industrial enzymes. It has to be experimentally determined in each individual case and the process adapted accordingly through the concentration and, optionally, dilution processes known per se.

As also shown in the EXAMPLES, this working range is preferably maintained throughout the process in order, on the one hand, for the process to operate at high concentrations and hence efficiently and, on the other hand, to lose as little enzyme as possible through denaturing and precipitation. In this way, yields of up to 95% can be obtained for the process as a whole.

Step (A) is optionally followed by the deodorization (A′) illustrated in FIG. 1, as discussed in the following.

The removal in step (B) of the precipitates (solids) formed by the concentration step applies in particular to foreign proteins and/or inactive enzymes, particularly in the vicinity of the solubility product. This step is designated as “separation” in the block flow chart of FIG. 1 and is also carried out in known manner, for example by means of a separator (see below), as also disclosed in the EXAMPLES of the present application.

The supernatant containing the target enzyme should be substantially free (see below) from suspended particles, i.e., solids, which—as mentioned above—can be determined by a tabletop centrifuge. This is because proteins precipitated as solids cannot be redissolved by dilution without considerable losses (see above) and without a drastic reduction in concentration. In addition, like other solids, they impair the following chromatography step by blocking the column.

Step (C), i.e., decoloration of the substantially solids-free supernatant of step (B) by means of a strongly basic anion exchanger (adsorber), represents the core of the invention. In this step, the colored impurities above all, more particularly the Maillard compounds, are adsorbed onto the resin while the proteins, which are positively charged under conditions to be correspondingly selected, are not bound to the resin by virtue of the strongly positive charge of the exchanger, but are obtained with the eluate in a substantially clear solution. Accordingly, step (C) represents a selective separation of the dyes from the concentrated enzyme solution.

The advantage of this process over the process described in the prior art is that the useful materials in question, i.e., the enzyme proteins, remain in solution, i.e., do not have to be denatured and renatured, and hence are not modified in their three-dimensional structure. Accordingly, they also remain in the phase to be further processed and are not discharged from the system, so that the high yields mentioned above can be achieved.

As indicated in FIG. 1 by corresponding thick arrows, the predominantly colored substances bound to the resin are subsequently eluted in a separate step, i.e., after discharge of the useful material phase (of the useful product) and optionally tailings. This is done, for example, with solutions of high ionic strength, for example concentrated NaCl solutions. The anion exchanger material can be regenerated by means of corresponding counterions, for example NaOH. Other simple salts may be more suitable, depending on the chromatography material. The fact that this material can be treated with such compounds, which are also inexpensive, results in sterilization in addition to the purifying effect. The system is thus suitable for cleaning in place (CIP).

After process step (C), the liquid enzyme is substantially free from troublesome streaks, precipitates and dyes. It remains light, clear and bright, even in the event of prolonged storage at various temperatures, while at the same time having a high level of stability. Examples of color values obtainable according to the internationally acceptable CIE color scale (defined in DIN 5033-3 and DIN 6174) can be found in the tests described in the EXAMPLES of the present application.

Process step (C) is optionally followed by step (D) described in detail hereinafter, in which the highly concentrated chromatography product is mixed with solvent. This is also illustrated in FIG. 1 (“mixing”).

The refined concentrated enzyme solution obtained after step (C) or (D) is clearly depleted, particularly in regard to the colored impurities, but still contains substantially colorless impurities, which are highly welcome by virtue of their partly stabilizing effect and which do not need, nor are intended, to be removed from the concentrated enzyme solution. Additional intermediate steps may be carried out in advance, inserted, added on or carried out together with the steps mentioned, depending on the separation problem. Examples of three such optional intermediate steps are described hereinafter.

Another possibility is, for example, selectively to remove other impurities from the concentrated enzyme solution by one or more additional chromatography steps, more particularly using other carrier materials which are adequately described in the prior art (see above). This may be done at any stage of the process which appears appropriate in each individual case, advantageously immediately before or after the chromatography step described in (C), optionally separated from one another by such intermediate steps as filtrations or re-solubilizations.

A change of solvent, which can be undertaken at various stages of the process according to the invention, preferably before or instead of step (D), is disclosed, for example, in German patent application DE 19953870 A1. This application describes a process for the production of substantially water-free enzyme preparation containing an organic solvent, in which an aqueous enzyme preparation is mixed with an organic solvent having a boiling point above 100° C. and the water is subsequently distilled off.

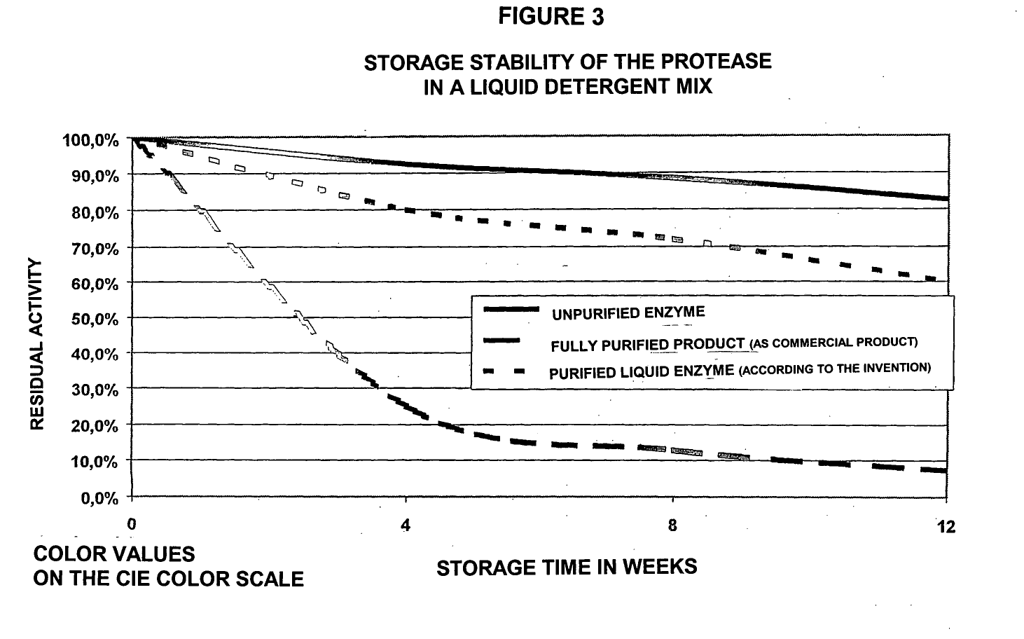

The liquid enzyme obtained by the process according to the invention may be used or further processed in known manner. It is of particular importance as a raw material for incorporation in detergents and cleaning compositions, more particularly in liquid form. The balance required in accordance with the invention between the clear color of such a product and the more than satisfactory stability is illustrated in EXAMPLE 3 of which the result is also shown in FIG. 3. It can be seen that the product refined in this way is only slightly more unstable than the unpurified enzyme, but is substantially colorless, and that, on the other hand, it is still very much more stable than a commercial product which has been conventionally decolored, i.e., by precipitation.

Preferred embodiments and other subjects of the invention are described in the following.

As mentioned above, enriched water-containing enzyme concentrates freed from the biomass by processes known per se described in the prior art are introduced into process step (A). Several component steps are generally required for this purpose. Preferred processes according to the invention are characterized in that an ultrafiltration is carried out as the last step immediately before process step (A), so that an ultrafiltration concentrate is introduced into step (A) according to the invention. One such process is described, for example in WO 01/37628 A2. Comparatively pure, low-solids enzyme solutions already enriched to a medium concentration value (cf. EXAMPLE 1) are obtained in this way.

As already mentioned, methods known per se, for example using a rotary evaporator or a thin-layer evaporator, preferably a thin-layer evaporator, are used for the concentration step (A).

In another preferred embodiment, step (A) is conducted by means of the particular parameters to be adjusted, more particularly the duration of the concentration process or optionally dilution, in such a way that an enzyme concentrate containing no more than 4 to 20% by weight, preferably no more than 4.5 to 15% by weight and, more particularly, no more than 5 to 10% by weight dry matter is obtained.

In contrast to the unwanted solids described above, the total solids content is the total content in the concentrated enzyme solutions of solid substances which would be obtained, for example, by complete evaporation of the solution. These values can be determined by methods known per se, for example by drying an aliquot or by absorption measurement and comparison with a calibration curve. The values mentioned have proved to be particularly suitable values for further processing because, on the one hand, the solution should be highly concentrated in order to avoid losses and because, on the other hand, an overly high viscosity would lead to difficulties in the constant throughput.

In another preferred embodiment, processes according to the invention are characterized in that, after step (A), the concentrated enzyme solution is deodorized in step (A′). Corresponding deodorization methods capable of being integrated into a continuous process are known from the prior art. They are particularly preferred when the microorganisms used for the preparation of the enzyme protein are microorganisms which form foul-smelling impurities or secrete proteins which degrade other constituents very rapidly.

Process step (B)—the separation of solids—which follows (A) or the deodorization step (A′) is also based on methods known per se, for example filtration. However, mechanical separation processes, preferably based on gravity or centrifugal separation, are preferred.

Such processes use, above all, separators, preferably continuous separators, which can be integrated into a continuous process. Separation processes involving the periodic discharge of sediment are particularly preferred. Such processes are also disclosed in the EXAMPLES of the present application.

The object of step (B) is to reduce the content of suspended material or solids in the enzyme concentrate to as low a value as possible. Preferred processes are characterized in that no more than 1% by volume, preferably no more than 0.7% by volume and more particularly no more than 0.5% by volume of solids is obtained in the concentrated enzyme solution by step (B). This can be adjusted through the known control of the particular equipment used. The above-mentioned separators in particular provide correspondingly advantageous solutions.

The core of the process according to the invention is step (C), i.e., strongly basic anion exchange chromatography. The success of the process is therefore critically dependent on the type of chromatography material selected and on the test procedure, for example sample application. As already mentioned, the colored impurities above all should be adsorbed onto the material while the proteins—positively charged under conditions to be correspondingly selected—are just not quite bound to the resin. Accordingly, the invention is based on a strongly basic anion exchanger. In view of the fact that most natural water-soluble, more particularly secreted proteins, are more soluble in water at medium pH values, it is of particular advantage for the strongly basic anionic exchanger for step (C) to develop maximum exchange capacity at a pH value of 5 to 9 and preferably 6 to 8.

Alkaline proteins in particular, such as for example the enzymes secreted by alkaliphilic microorganisms, more especially proteases, have an isoelectric point in the alkaline range and are therefore positively charged in the preferred pH range and, hence, do not bind to the particular material. As already mentioned, an ideal pH for this process has to be experimentally determined for each protein and adjusted with regard to this step (C). In EXAMPLES 1 and 2, these values were ca. 7.5 and 7 for the alkaline protease selected and the α-amylase selected.

Strongly basic anion exchangers containing quaternary ammonium groups as functional groups, preferably those substituted by at least two alkyl groups and more preferably those substituted by at least two C₁ or C₂ alkyl groups and optionally by a C₁ or C₂ hydroxyalkyl group, have proved to be particularly suitable for step (C) by virtue of their chemical properties.

This requirement is satisfied, in particular, by strongly basic anion exchangers containing the functional groups trimethyl ammonium or dimethyl ethanol ammonium. The latter is a little more weakly basic than the former, so that corresponding proteins can be optimized in particular through this variation.

Accordingly, corresponding chromatography materials characterize preferred embodiments.

Another characteristic of chromatography materials is their exchange capacity, which is expressed in mol equivalent per unit volume. It indicates how densely the material is occupied by the functional groups. Particularly suitable processes are characterized in that the strongly basic anion exchanger for step (C) has an exchange capacity of 0.7 to 1.2 meq/ml, preferably 0.8 to 1.1 meq/ml and more particularly 0.9 to 1.0 meq/ml.

Another criterion which influences the separation efficiency of chromatography columns is the effective pore size. It has to be gauged in such a way that retention is adequate without the substances flushed through, more particularly the proteins, being too strongly retained or even blocking the material. A chromatography material with an effective pore size of 0.45 mm proved to be suitable for the two globular proteins investigated in the EXAMPLES, B. lentus alkaline protease and α-amylase from Bacillus sp. A 7-7 (DSM 12368), which have molecular weights of ca. 27 kD and ca. 58 kD, respectively. For distinctly larger or smaller proteins, the chromatography materials selected should have correspondingly larger or smaller effective pore sizes.

Accordingly, preferred processes are characterized in that the strongly basic anion exchanger has effective pore sizes of 0.2 to 0.7 mm, preferably 0.3 to 0.6 mm and more preferably 0.4 to 0.5 mm.

In principle, suitable carriers for strongly basic anion exchangers to be used in accordance with the invention are any of the materials described for this purpose in the prior art including, for example, gel-form carriers. By contrast, strongly basic anion exchangers based on a porous polymer are preferred for step (C) by virtue of their technical properties. Strongly basic anion exchangers based on a styrene/DVB copolymer have proved to be particularly advantageous.

Chromatography materials having the properties just discussed are described in detail in the prior art. Those from the DIAION® series are described, for example, in the manual “DIAION®, Manual of ion exchange resins and synthetic adsorbent, Vol. 1”, Mitsubishi Kasei Corp. (Tokyo, Japan), June 1995, pp. 104 to 108 and in “Product Line Brochure DIAION®” 1.6.2001, pp. 4 to 6, which is obtainable from the manufacturer or from Summit Chemicals Europe GmbH, Düsseldorf, Germany. Strongly basic anion exchangers described there include the series DIAION® SA, DIAION® PA and DIAION® HPA. One representative, namely DIAION® PA308L, was successfully used in the EXAMPLES of the present application.

Chemically similar materials which may be used for the chromatography step (C) can be produced by relevant experts in accordance with the foregoing observations on preferred properties or may also be obtained from other commercial manufacturers and, again, characterize preferred embodiments. Comparable results are obtained, for example, with the materials DOW MSA Marathon® from Dow Chemicals and Amberlite® 900CL from Rohm & Haas.

The chromatography step (C) is advantageously carried out under certain conditions. These include in particular a certain bed volume, which is the ratio by volume of the substance applied to that of the column, and a certain residence time which is favorably expressed by means of the enzyme solution.

It has proved to be particularly advantageous and characterizes correspondingly preferred embodiments to carry out step (C) with a bed volume of 1 to 10, preferably 1.5 to 7 and more preferably 2 to 4. These bed volumes represent the optimum experimentally determined in the EXAMPLES for keeping the filtrate as clean and at the same time as concentrated as possible.

Suitable average residence times in step (C), which characterize correspondingly preferred embodiments, are values of 0.01 to 0.2 g, preferably 0.025 to 0.1 g, more preferably 0.04 to 0.06 g and most preferably 0.05 g enzyme per g carrier material per minute.

Particularly suitable processes are characterized in that they are controlled largely automatically. A control method which is particularly easy to incorporate is based on determining the conductivity (measurable as μS/cm) of a processed material at critical places and using the results to control the process. This is possible, for example, after the chromatography phase and may be used to separate the fractions containing useful material (useful product) from the others. Accordingly, in one preferred embodiment, the process according to the invention is characterized in that step (C), more particularly the separation between forerun and useful product and/or useful product and tail, is controlled through the conductivity of the eluate.

In order to increase yield, it had already been proposed in patent application WO 01/37628 A2 to use filtrate for an additional washing step. Accordingly, processes according to the invention are also preferably characterized in that step (C) is carried out with recycling of at least part of the forerun and/or the tail of the ion exchange chromatography. In this way, the fraction in question is additionally enriched with enzyme molecules which had still not been washed out from the column towards the end of the peak. This step is limited in principle by the possible impurities which are possibly washed out from the column. In each individual case, a balance has to be struck between the concentration obtainable and product quality, i.e., purity.

During the process described thus far, a high enzyme concentration has been used in the interests of efficiency. However, concentrated enzyme solutions are often not needed in such high concentrations for their particular industrial application. Accordingly, correspondingly preferred processes according to the invention are characterized in that, after step (C), a relatively low concentration is adjusted by dilution in a step (D). Mixers known from the prior art, more particularly types which can be integrated into a continuous system, are used for this purpose.

On the other hand, all liquid enzymes have a tendency in storage to denature and thus to lose their activity. This applies in particular to proteases which hydrolyze other enzyme molecules. Accordingly, a preferred process according to the invention is characterized in that a stabilizer or a stabilizer mixture is added after step (C). Such compounds are known per se from the prior art and include, for example, compounds which develop a stabilizing effect, for example against temperature variations, biophysically through regulation of the water activity, such as polyols, and compounds which reversibly deactivate proteases or which afford protection against oxidation.

The stabilizer or stabilizer mixture may optionally be added before, after or at the same time as a dilution (D). In a particularly advantageous embodiment, step (D) is used to add a stabilizer solution together with the diluting solution or a solution which exerts both effects.

The stabilizer added is preferably selected from liquid compounds containing hydroxyl groups, for example a polyol, such as glycerol or, in a particularly preferred embodiment, propane-1,2-diol. The liquid compounds in question may also be mixtures with water and/or other stabilizing compounds.

In order to develop the stabilizing effect, it has proved to be particularly favorable to add the polyol stabilizer mixture in a quantity of 40 to 70% by volume, preferably 45 to 65% by volume and more preferably 50 to 60% by volume, based on the final volume.

If dilution were to result in a too weakly concentrated solution at this point, a concentration step could optionally be inserted between steps (C) and (D) before the solution is passed through the mixer. In principle, any methods known from the prior art, preferably the methods described above, may be used for this purpose.

This is also another argument in support of using a highly concentrated enzyme solution in the hitherto known process in order, through this drastic diluting effect, to obtain a liquid enzyme which is still highly concentrated enough for the industrial applications envisaged.

This dilution step may also be used to adjust the end product of the process to a total solids content of 2 to 15% by weight, preferably 5 to 13% by weight and more preferably 8 to 12% by weight. It may also be used to adjust the end product of the process to a viscosity value of 1 to 20 mPas, preferably 1 to 15 mPas and more preferably 1 to 10 mPas at 25° C. and/or to a sediment content of less than 1% by volume, preferably less than 0.75% by volume and more preferably less than 0.5% by volume. These values generally correlate with one another and have proved to be particularly suitable for the further storage and/or handling of liquid enzymes. As mentioned above, this adjustment has to be taken into account after the chromatography phase or before addition of a stabilizer. Accordingly, processes according to the invention which meet these requirements are preferred.

The present specification refers to enzymes of which concentrated solutions are refined by processes according to the invention. Enzymes represent preferred embodiments because, on the one hand, they are of particular industrial interest and, on the other hand, can be detected through their specific activities which can be used in particular for determining the optimum working range according to EXAMPLES 1 and 2 and FIG. 2. Nonetheless, this process may be applied to any water-soluble proteins providing suitable solvent systems and chromatography materials can be found for them and suitable detection reactions can be established. This applies, for example, to peptides, for example peptide hormones, pharmacologically significant oligopeptides, and to antibodies. Antibodies would also be suitable, for example, for detection. All these proteins are intended to be understood as enzymes in the context of the present invention.

However, industrially useful enzymes in the conventional sense are the focus of interest, preferably a hydrolase or an oxidoreductase and more preferably a protease, amylase, cellulase, hemicellulase, lipase, cutinase or a peroxidase. Processes according to the invention designed in particular for processing such enzyme solutions through the determination and establishment of suitable process conditions (see above) represent correspondingly preferred embodiments of the present invention.

Proteases are of major interest, more particularly for the production of detergents and cleaning compositions, alkaline proteases being preferred because they are particularly active and can be incorporated in alkaline formulations. Accordingly, correspondingly preferred processes are those characterized by suitable proteases.

Among such processes, those which are characterized in that step (C) in particular is carried out at a pH value of 5 to 9, preferably 6 to 8.5 and more preferably 7 to 8 are preferred by virtue of the biochemical properties of the alkaline proteases mentioned.

One such protease was also investigated in EXAMPLES 1 and 3 where it was shown that maintaining a critical activity value is necessary for carrying out a process according to the invention in order to keep the level of solids occurring to a minimum. Accordingly, correspondingly preferred processes for refining the concentrated protease solutions mentioned are characterized in that the product of step (A) is adjusted to an activity value of 600,000 to 900,000, preferably 650,000 to 850,000 and more preferably 700,000 to 800,000 HPU per g. The parameters of the above-mentioned component steps, as known from the prior art, are suitable for this adjustment, i.e., concentration or optionally dilution.

In accordance with the foregoing observations, the end products intended for subsequent use advantageously have relatively low activity values which can be established in particular by dilution with stabilizing solutions. Based on proteases, processes by which the end product is adjusted to an activity value of 150,000 to 500,000, preferably 175,000 to 300,000 and more preferably 200,000 to 260,000 HPU per g represent preferred embodiments.

Another industrially important enzyme type are the α-amylases. They are used, for example, in the food industry for the production of confectionery or, by virtue of their starch-hydrolyzing activity, are added to detergents and cleaning compositions. As with the proteases, alkaline α-amylases are particularly preferred. Accordingly, correspondingly preferred processes are those which are designed for processing α-amylases and which are characterized by them, preferably for, or by, those with an alkaline pH optimum.

As stated in EXAMPLES 2 and 3, preferred processes according to the invention for processing α-amylases are characterized in that the product of step (A) is adjusted to an activity value of 30,000 to 50,000 TAU per g and preferably 35,000 to 45,000 TAU per g.

Since α-amylases are also generally used in correspondingly lower concentrations and, in addition, should also be stabilized, other preferred embodiments of the process according to the invention are characterized in that the end product is adjusted to an activity value of 4,000 to 14,000, preferably 6,000 to 12,000 and more preferably 8,000 to 10,000 TAU per g.

Another particularly important enzyme type are cellulases which are used, for example, in the detergent industry and in textile production for the surface treatment of textiles. Accordingly, processes which are characterized by a cellulase, preferably with an alkaline pH optimum, represent correspondingly preferred embodiments of the present invention.

All the process parameters mentioned thus far are reflected in the particular process products, for example in regard to the type of enzyme, the purity level, the nature of the compounds separated off and the activity or stability values obtained. Accordingly, the products obtained by these processes according to the invention are also preferred. Accordingly, besides the process, the present invention also relates to concentrated enzyme solutions which have been obtained by a process described in the foregoing.

The present invention also relates to compositions containing an enzyme which has been obtained as an intermediate product in the form of a concentrated enzyme solution according to the invention. For example, the concentrated enzyme solutions obtained in accordance with the invention do not have to be used in liquid form, but instead may be converted into a dry, highly pure form. This can be done, for example, by freeze-drying or by incorporation in solid granules. Corresponding processes are described in detail in the prior art. In this form, they can be stored for long periods or incorporated in other solid compositions, for example in solid detergents and cleaning compositions.

Accordingly, this embodiment of the invention also includes all the various possible types of cleaning compositions—both concentrates and compositions to be used without dilution—for use on a commercial scale in washing machines or in hand washing or cleaning. These include, for example, detergents for fabrics, carpets or natural fibers, for which the term “detergent” is used in the present specification; dishwashing detergents for dishwashing machines or manual dishwashing detergents or cleaners for hard surfaces, such as metal, glass, china, ceramic, tiles, stone, painted surfaces, plastics, wood or leather, for which the term “cleaning composition” is used in the present specification. Any type of detergent or cleaning composition represents an embodiment of the present invention providing it is enriched by an enzyme which has been refined by the process according to the invention and further processed in accordance with this particular aspect of the invention.

Embodiments of the invention encompass all well-known and/or appropriate supply forms of the detergents or cleaning compositions according to the invention. These include, in particular, solid powder-form compositions, optionally of several phases, compressed or uncompressed; extrudates; granules; tablets or pouches, packed both in large containers or in portions. Liquid, paste-form or gel-form embodiments are also included providing the enzyme processed in accordance with the invention is introduced in a further-processed form for such embodiments.

In a preferred embodiment, the detergents or cleaning compositions according to the invention contain active enzymes in a quantity of 2 μg to 20 mg, preferably 5 μg to 17.5 mg, more preferably 20 μg to 15 mg and most preferably 50 μg to 10 mg per gram of the composition.

Besides an enzyme prepared in accordance with the invention and possibly other enzymes, a detergent or cleaning composition according to the invention optionally contains other ingredients such as, for example, enzyme stabilizers, surfactants, for example nonionic, anionic and/or amphoteric surfactants, bleaching agents, bleach activators, bleach catalysts, builders, solvents, thickeners and—optionally as further typical ingredients—sequestering agents, electrolytes, optical brighteners, redeposition inhibitors, dye transfer inhibitors, foam inhibitors, dyes and/or perfumes, antimicrobial agents and/or UV absorbers to mention only the most important classes of ingredients. Corresponding formulations are described in detail in the prior art.

By contrast, compositions which contain a suitably concentrated enzyme solution are preferred by virtue of the advantageous properties of the liquid enzymes obtained in accordance with the invention and particularly by virtue of their clear appearance and because they can be used without further working up. Compositions present as a whole in liquid, paste or gel form are of particular interest. They are easy to dose, contain the enzyme in the required activity and are attractive in appearance, at least so far as the enzyme component is concerned. This was emphasized as desirable at the beginning.

This applies in particular to detergents and cleaning compositions designed for the end user. Accordingly, in one particularly preferred form, the compositions of this embodiment are detergents or cleaning compositions. These come under the above definition and may contain the substances mentioned there. In addition, in this embodiment, the compositions as a whole have a liquid, gel-like or paste-like consistency in which the refined products according to the invention may readily be incorporated, i.e., by methods known per se.

### EXAMPLES

### Example 1

### Refining a Concentrated Protease Solution

Separating the Biomass

After production of the useful material, protease, by fermentation as described in WO 91/02792 A1, the biomass was almost completely separated by the known methods of separation, microfiltration and sterile filtration. This was followed, as described in WO 01/37628 A2, by concentration, i.e., removal of the solvent by ultrafiltration until the protease concentrate had an activity of 300,000 to 400,000 HPU per g as determined by the method described in the article by van Raay, Saran and Verbeek entitled “Zur Bestimmung der proteolytischen Aktivität in Enzymkonzentraten und enzymhaltigen Wasch-, Spül- und Reinigungsmitteln [Determining Proteolytic Activity in Enzyme Concentrates and Enzyme-containing Laundry Detergents, Dishwashing Detergents and Cleaners]” in Tenside (1970), Vol. 7, pp. 125-132. In addition, a pH of 7.5 was adjusted with a 30% CaCl₂ solution. The solution had a solids content of less than 1% by volume, as determined by centrifuging with a tabletop or laboratory centrifuge for 10 mins. at 7,000 G. In addition, the protease obtained showed a positive charge for an ionic strength of 1 to 20 mS/cm.

Determining the Optimal Working Range

Samples of the solution obtained after separation of the biomass were taken during the ultrafiltration step (values up to 400,000 HPU) or were further concentrated by means of a separator, as described below, and the solids content was determined in dependence upon the particular activity, as stated above. The activity measurements were carried out at a pH of 7.5, at a temperature of 20° C. and at an ionic strength of 10 mS/cm. The dependence of the solids content on the concentration of active protease is illustrated in TABLE 1 and in FIG. 2.

TABLE 1
Dependence of the Solids Content on the Concentration of Active
Protease

| | Solids content |
|---|---|
| Activity | of the solution |
| [1000 HPU/g] | [% by vol.] |
| 0 | 0.2 |
| 200 | 0.5 |
| 400 | 1.2 |
| 600 | 1.5 |
| 800 | 3.0 |
| 1000 | 7.5 |

As can be seen from TABLE 1, the solids content of a concentrated protease solution increases overproportionally beyond ca. 900,000 HPU/g, so that the range from ca. 700,000 to 800,000 HPU/g may be regarded as the optimal working range with maximal activity and a minimal solids content.

Step (A): Concentration of the Enzyme Solution to the Working Range

On the basis of this result, the enzyme solution ultimately obtained by ultrafiltration in the preceding step was concentrated to a value of 800,000 HPU/g in a thin-layer evaporator under the following conditions: temperature of the product above 35° C., vacuum of ca. 20 mbar, substantially constant pH of ca. 7.5 and a solids content of the enzyme concentrate kept at less than 3% by volume, so that the losses in the following step remain minimal.

Step (B): Separation of the Precipitates (Solids) Formed

Separation of the solids (precipitates) formed by concentration was carried out by mechanical separation on the basis of the principle of gravity or centrifugal separation. A separator with periodic discharge of sediment was used to separate the solids (ALFA LAVAL BTPX 205 with an Σ value of 11,700 m² operated at a G value of 12,800 and a throughput of 200 l/h). A largely solids-free enzyme concentrate was thus obtained (solids content less than 0.2% by volume determined as described above). The activity remained at ca. 800,000 HPU/g.

Step (C): Strongly Basic Anion Exchange Chromatography

Chromatographic decoloring was carried out in a fixed bed using a strongly basic anion exchanger of the DIAION® Pa 308 L type obtainable from Mitsubishi, Tokyo, Japan or from Mitsubishi Chemical Europe GmbH, Düsseldorf, Germany. The decoloring quality and hence the stability of the enzyme were controlled through the bed volume ratio (BV—ratio by volume of the enzyme concentrate to the resin) and the residence time. A ratio of 2 to 5 BV and a dosage of 0.05 kg enzyme solution per kg resin per min. were adjusted. The principle was based on the enzyme being repelled by the carrier material and entrained into the liquid stream while the dyes bind to the immobile carrier. In order to increase the yield, part of the tail was passed through the column again. The compounds adsorbed onto the column, more particularly the dyes, were then washed out by flushing with NaCl and NaOH solutions and the fixed bed was regenerated in this way.

Step (D): Mixing, Stabilization and Activity Adjustment with Solvent

The filtrate with an activity value of ca. 700,000 HPU per g was directly mixed, i.e., on line in a static mixer, with propane-1,2-diol which, at the same time, has a stabilizing effect. Ca. 55% by volume of solvent were taken.

The liquid enzyme refined through these four steps had the following properties (activity was determined as defined above and color was determined using the internationally adopted CIE color scale defined in DIN 5033-3 and DIN 6174):
Activity: 260,000 HPU/g
Color: L value>96
   b* value<14
Viscosity: <10 mPas
pH value: ca. 7

The liquid obtained was substantially clear and did not show any precipitate immediately after the process. The further stability is described in EXAMPLE 3.

### Example 2

### Refining a Concentrated Amylase Solution

An amylase solution refined in accordance with the invention was prepared as in EXAMPLE 1 except for the following differences. A fermenter batch containing as useful material the α-amylase described in application WO 02/01036 A2 was used. Since the α-amylase has a different isoelectric point from protease, a pH of 6.25 was adjusted before step (A) and a pH of 6 to 6.5 was maintained throughout the process to keep the charge of the amylase positive.

Determination of Activity

The amylolytic activity in TAU was determined using a modified p-nitrophenyl maltoheptaoside of which the terminal glucose unit was blocked by a benzylidene group; the p-nitrophenyl maltoheptaoside was split by amylase into free p-nitrophenyl oligosaccharide which in turn was converted with the auxiliary enzymes glucoamylase and α-glucosidase into glucose and p-nitrophenol. The quantity of p-nitrophenol released was thus proportional to the amylase activity. The measurement was carried out, for example, with an Abbott Quick-Start® test kit (manufacturer: Abbott, Abbott Park, Ill., USA). The increase in absorption (405 nm) in the test mixture was detected by photometer against a blank value over a period of 3 mins. at 37° C. Calibration was based on an enzyme standard of known activity (for example Maxamyl®/Purastar® 2900 from Genencor, Palo Alto, Calif., USA, with 2,900 TAU/g). Evaluation was carried out by plotting the difference in absorption dE (405 nm) per min. against the enzyme concentration of the standard.

Determination of the Optimal Working Range

As in EXAMPLE 1, samples differing in concentration were taken during the ultrafiltration step and the subsequent separation and the solids content was again determined in dependence upon the particular activity. The measurements were carried out at a pH of 6.25, at a temperature of 20° C. and at an ionic strength of 10 mS/cm. The results are shown in TABLE 2 and in FIG. 2.

TABLE 2
Dependence of the Solids Content on the Concentration of Active
α-amylase

| | Solids content of |
|---|---|
| Activity | the solution |
| [100 TAU/g] | [% by vol.] |
| 0 | 0.5 |
| 100 | 1.0 |
| 200 | 1.5 |
| 300 | 2.5 |
| 400 | 3.2 |
| 500 | 5.0 |

As can be seen from TABLE 2, amylase is comparable with protease (see above) in the dependence of the solids content on the concentration of active enzyme; this is expressed in FIG. 2 in 100 TAU per g. Accordingly, the solids content of a concentrated amylase solution increases overproportionally above ca. 50,000 TAU/g, so that the range from ca. 35,000 to 45,000 TAU/g may be regarded as the optimal working range with maximal activity and a minimal solids content. Accordingly, the working range according to the invention should be below that range.

Accordingly, an activity value of 35,000 to 45,000 TAU per g was adjusted through step (A) carried out as in EXAMPLE 1 and the further procedure was carried out as in EXAMPLE 1, i.e., using the same anion exchange chromatography material. In step (D), a concentration value of 9,000 TAU per g was similarly adjusted by mixing with propane-1,2-diol.

The liquid enzyme refined by these steps had the following properties:
Activity: 9,000 TAU/g
pH: ca. 6.25

The liquid was substantially clear and, like the protease in EXAMPLE 1, did not show any precipitate immediately after the process.

### Example 3

### Storage Stability of the Protease in a Liquid Detergent Matrix

To determine the storage stability of a protease refined according to the invention by comparison with the unpurified enzyme and a commercial product in the same matrix of liquid detergent, the following three samples were prepared: (1.) an unrefined protease as present after ultrafiltration and before step (A) in EXAMPLE 1, (2.) the fully purified commercial product Savinase® 16.0 LEX obtainable from Novozymes, Bagsvaerd, Denmark and (3.) the protease refined in accordance with EXAMPLE 1. All three samples were placed in an activity of 260,000 HPU per g in a solution of 55% by volume propane-1,2-diol in water and incorporated in a quantity of 0.4% by volume in a liquid detergent matrix of typical composition.

The protease samples introduced into this matrix had L values on the CIE color scale (see above) of (1.) 78, (2.) 99 and (3.) 97. In other words, the protease refined in accordance with the invention was almost as clear as the fully purified product. Samples were taken at regular intervals over a period of 12 weeks and the residual activities were determined as described above. The values set out in TABLE 3 and shown as a graph in FIG. 3 were obtained.

TABLE 3
Storage Stability of the Protease in a Liquid Detergent Matrix
(Expressed as the Residual Activity of HPU in %)

| Sample | 0 Weeks | 4 Weeks | 8 Weeks | 12 Weeks |
|---|---|---|---|---|
| Unrefined protease | 100 | 92.5 | 88.5 | 82.5 |
| Savinase ® | 100 | 25.0 | 12.8 | 7.3 |
| 16.0 LEX | | | | |
| Protease refined in | 100 | 80.0 | 72.0 | 60.0 |
| accordance with | | | | |
| EXAMPLE 1 | | | | |

It can be seen that the protease refined in accordance with the invention, despite virtually the same color value, is much more stable than the fully purified product and that the protease refined in accordance with the invention loses only a little activity in a detergent matrix as compared with the dark unpurified enzyme.

1. A process for refining concentrated enzyme solutions comprising the steps of: (a) providing a concentrated enzyme solution comprised of insoluble solids; (b) separating the insoluble solids to produce a solids-free supernatant solution and (c) contacting the supernatant solution with a strongly basic anion exchanger carried out with a bed volume of from 1 to 10 to produce a decolorized protein solution.

2. The process of claim 1 wherein the concentrated enzyme solution of step (A) is concentrated by ultrafiltration.

3. The process of claim 1 wherein the concentrated enzyme solution of step (A) is concentrated by a thin-layer evaporator.

4. The process of claim 1 wherein the solids content of the concentrated enzyme solution is from 4 to 20% by weight

5. The process of claim 1 wherein the concentrated enzyme solution of step (A) is deodorized prior to step (B).

6. The process of claim 1 wherein the insoluble solids are separated in step (B) by centrifugation.

7. The process of claim 1 wherein step (B) is accomplished in a continuous separator with periodic sample discharge.

8. The process of claim 1 wherein the solids content of the solution after step (A) is no more than 1% by volume.

9. The process of claim 1 wherein the pH range of the maximum exchange capacity of the anion exchanger is from 5 to 9.

10. The process of claim 1 wherein the anion exchanger is comprised of quaternary ammonium functional groups optionally substituted by a C₁ or C₂ hydroxyalkyl group.

11. The process of claim 10 wherein the quaternary ammonium functional groups are substituted by at least two alkyl groups.

12. The process of claim 1 wherein the alkyl groups C₁ or C₂ alkyl groups.

13. The process of claim 1 wherein the anion exchanger is comprised of trimethyl ammonium or dimethylethanol ammonium groups functional groups.

14. The process of claim 1 wherein the exchange capacity of the anion exchanger is from 0.7 to 1.2 meq/ml.

15. The process of claim 1 wherein the anion exchanger has an effective pore size of from 0.2 to 0.7 mm.

16. The process of claim 1 wherein the anion exchanger is comprised of a porous styrene/DVB copolymer.

17. The process of claim 1 wherein the average time that the supernatant is in contact with the anion exchanger is from 0.01 to 0.2 g of enzyme per gram of carrier material per minute.

18. The process of claim 1 wherein the separation between the forerun and useful product in step (C) is identified by the conductivity of the eluate.

19. The process of claim 1 wherein at least part of the forerun and/or tail of the eluate is recycled in step (C).

20. The process of claim 1 further comprising step (D) wherein step (D) is comprised of diluting the decolorized protein solution formed in step (C).

21. The process of claim 20 wherein a stabilizer is added before step (D).

22. The process of claim 20 wherein a stabilizer is added during step (D).

23. The process of claim 21 wherein a stabilizer is a polyol.

24. The process of claim 23 wherein a stabilizer is propane-1,2-diol.

25. The process of claim 22 wherein the amount of the stabilizer is from 40 to 70% by volume.

26. The process of claim 1 wherein the dry matter content of the decolorized protein solution is adjusted to 2 to 15% by weight.

27. The process of claim 26 wherein the viscosity of the decolorized protein solution is adjusted to a viscosity of 1 to 20 mPas.

28. The process of claim 1 wherein the sediment content of the decolorized protein solution is adjusted to less than 1% by volume.

29. The process of claim 1 wherein the enzyme is selected from the group consisting of a hydrolase, an oxidoreductase, a protease, an amylase, a cellulase, a hemicellulase, a lipase, a cutinase and a peroxidase.

30. The process of claim 29 wherein the enzyme is an alkaline protease.

31. The process of claim 1 wherein step (C) is carried out at a pH of from 5 to 9.

32. The process of claim 1 wherein the activity of the product of step (A) is adjusted to from 600,000 to 900,000.

33. The process of claim 1 wherein the activity of the decolorized protein solution is adjusted to from 150,000 to 500,000 HPU per g.

34. The process of claim 1 wherein the activity of the decolorized protein solution is adjusted from 200,000 to 260,000 HPU per g.

35. The process of claim 29 wherein the enzyme is an α-amylase with an alkaline pH optimum.

36. The process of claim 29 wherein the product of step (A) is adjusted to an activity of 30,000 to 50,000 TAU per g.

37. The process of claim 1 wherein the activity of the decolorized protein solution is adjusted to from 4,000 to 14,000 TAU per g.

38. The process of claim 29 wherein the enzyme is a cellulose with an alkaline pH optimum.

39. A concentrated enzyme solution which is the product of the process of claim 1.

40. An isolated, dry, highly pure enzyme which is the product of the process of claim 1.

41. A composition containing the enzyme of claim 40 wherein the composition is in the form of a liquid, paste or gel form.

42. An improved detergent or cleaning composition comprising detergent or a cleaning composition of claim 41.
